(19) United States
(12) Patent Application Publication
(10) Pub. No.: US 2019/0216902 A1
(43) Pub. Date: Jul. 18, 2019

(54) AMINO ACID COMPOSITION FOR COLLAGEN FORMATION

(71) Applicants: Joseph Cheng Lin, Walnut, CA (US); Ivy Ewei Lin, Walnut, CA (US)

(72) Inventors: Joseph Cheng Lin, Walnut, CA (US); Ivy Ewei Lin, Walnut, CA (US)

(21) Appl. No.: 16/231,712

(22) Filed: Dec. 24, 2018

Related U.S. Application Data
(60) Provisional application No. 62/618,062, filed on Jan. 16, 2018.

(51) Int. Cl. A61K 38/39; A61K 31/198

(52) U.S. Cl. CPC A61K 38/39; A61K 31/198

(57) ABSTRACT

An amino acid composition for collagen formation includes a first chain unit component, a second chain unit component, and a third chain unit component of collagen. The first chain unit component, the second chain unit component, and the third chain unit component are the same or different from each other, and each of the first chain unit component, the second chain unit component, and the third chain unit component is selected from the group consisting of al chain component, α2 chain component, α3 chain component, α4 chain component, α5 chain component, and α6 chain component. The amino acid composition includes Alanine, Phenylalanine, Cysteine, Aspartate, Asparagine, Glutamate, Glutamine, Glycine, Histidine, Leucine, Isoleucine, Lysine, Methionine, Proline, Arginine, Serine, Threonine, Valine, Tryptophan, and Tyrosine and/or the pharmaceutically acceptable salt or ester derivatives thereof.

### CROSS-REFERENCE TO RELATED PATENT APPLICATION

This application claims priority from the U. S. Provisional Patent Application Ser. No. 62/618,062 filed Jan. 16, 2018, which application is incorporated herein by reference in its entirety.

Some references, which may include patents, patent applications and various publications, may be cited and discussed in the description of this disclosure. The citation and/or discussion of such references is provided merely to clarify the description of the present disclosure and is not an admission that any such reference is “prior art” to the disclosure described herein. All references cited and discussed in this specification are incorporated herein by reference in their entireties and to the same extent as if each reference was individually incorporated by reference.

### FIELD OF THE DISCLOSURE

The present disclosure relates to an amino acid composition for collagen formation, more particularly an amino acid composition which (1 ) can be directly absorbed and utilized without gastrointestinal digestion, (2) can avoid allergic reactions in consumers with allergens from specific sources, and (3) is suitable for ingestion by vegetarians.

### BACKGROUND OF THE DISCLOSURE

Collagen is a main structural protein of various connective tissues in animals. As the main component of connective tissue, it is the most abundant protein in mammalian proteins. Collagen accounts for 25 %-35 % of the total protein content, and is widely present in skin, muscle, cartilage, ligaments, tendons, bones, teeth and other tissues.

The amino acid composition of collagen includes two uncommon amino acids: hydroxyproline and hydroxylysine. These two amino acids are formed during protein post-translational modification. In other words, proline and lysine of propeptide form hydroxyproline and hydroxylysine under the action of prolyl hydroxylase, lysyl hydroxylase and vitamin C, respectively. Accordingly, the two amino acids hydroxyproline and hydroxylysine are not required as raw materials in the process of producing collagen in the human body.

Conventional collagen health supplements in the existing market can be roughly classified into two generations: collagen protein (e.g. type I collagen) as the first-generation of collagen health supplements and collagen peptide (e.g. type I collagen peptide) as the second-generation of collagen health supplements. The main sources of these collagen health supplements are fish or animals (such as cattle or chicken). Further, the second-generation type I collagen peptide is a peptide that hydrolyzes type I collagen into smaller molecules, which is easier to be digested and absorbed than the first-generation type I collagen. However, both the first-generation type I collagen and the second-generation type I collagen peptide are digested by the digestive system to produce hydroxyproline and hydroxylysine, as well as dipeptides or tripeptide thereof, which are not raw materials necessary for synthesizing collagen. Therefore, these two amino acids should not be used to synthesize collagen, as the two amino acids have no substantial benefit on the proliferation of human collagen.

Given that commercial collagen supplements are mainly of animal source, e.g. fish or domesticated animals such as cattle or chickens, the collagen would not be suitable for consumers who are allergic to specific animal sources, and consumers who are vegetarians.

In view of this, there is still a need for a collagen health supplement that can avoid allergies in consumers who are allergic to specific sources, are suitable for vegetarians, and further can be effectively used by the human body.

### SUMMARY OF THE DISCLOSURE

In response to the above-referenced technical inadequacies, the present disclosure provides an amino acid composition for collagen formation, which can be directly sent to the blood through the small intestine, and then transferred to other tissue cells without being digested by the intestines, so as to be directly absorbed and utilized to synthesize collagen. Since the amino acid composition of the present disclosure consists directly of amino acids, it can not only avoid allergic reactions in consumers who are allergic to specific sources, but also be consumed by vegetarians.

In one aspect, the present disclosure provides an amino acid composition for collagen formation, including a first chain unit component, a second chain unit component, and a third chain unit component. The first chain unit component, the second chain unit component and the third chain unit component are the same or different from each other, whereby each component is selected from the group consisting of al chain component, α2 chain component, α3 chain component, α4 chain component, α5 chain component and α6 chain component. The al chain component, α2 chain component, α3 chain component, α4 chain component, α5 chain component and α6 chain component each includes Alanine, Phenylalanine, Cysteine, Aspartate, Asparagine, Glutamate, Glutamine, Glycine, Histidine, Leucine, Isoleucine, Lysine, Methionine, Proline, Arginine, Serine, Threonine, Valine, Tryptophan, and Tyrosine and/or the pharmaceutically acceptable salt or ester derivatives thereof.

One of the advantages of the present disclosure is that the amino acid composition for collagen formation can utilize the technical features of “a first chain unit component, a second chain unit component and a third chain unit component each being selected from the group consisting of al chain component, α2 chain component, α3 chain component, α4 chain component, α5 chain component and α6 chain component” and “an al chain component, α2 chain component, α3 chain component, α4 chain component, α5 chain component and α6 chain component each including Alanine, Phenylalanine, Cysteine, Aspartate, Asparagine, Glutamate, Glutamine, Glycine, Histidine, Leucine, Isoleucine, Lysine, Methionine, Proline, Arginine, Serine, Threonine, Valine, Tryptophan, and Tyrosine and/or the pharmaceutically acceptable salt or ester derivatives thereof” to achieve the beneficial effect of the present disclosure.

Specifically, the amino acid composition for collagen formation of the present disclosure can be absorbed directly and utilized by organisms to achieve the purpose of synthesizing collagen. Further the amino acid composition can avoid allergies in consumers who are allergic to specific sources and is suitable for vegetarians.

These and other aspects of the present disclosure will become apparent from the following description of the embodiment taken in conjunction with the following drawings and their captions, although variations and modifications therein may be affected without departing from the spirit and scope of the novel concepts of the disclosure.

### BRIEF DESCRIPTION OF THE DRAWINGS

The present disclosure will become more fully understood from the following detailed description and accompanying drawings.

FIG. 1 is a flowchart of preparing the amino acid composition for collagen formation according to an embodiment of the present disclosure.

### DETAILED DESCRIPTION OF THE EXEMPLARY EMBODIMENTS

The present disclosure is more particularly described in the following examples that are intended as illustrative only since numerous modifications and variations therein will be apparent to those skilled in the art. Like numbers in the drawings indicate like components throughout the views. As used in the description herein and throughout the claims that follow, unless the context clearly dictates otherwise, the meaning of “a”, “an”, and “the” includes plural reference, and the meaning of “in” includes “in” and “on”. Titles or subtitles can be used herein for the convenience of a reader, which shall have no influence on the scope of the present disclosure.

The terms used herein generally have their ordinary meanings in the art. In the case of conflict, the present document, including any definitions given herein, will prevail. The same thing can be expressed in more than one way. Alternative language and synonyms can be used for any term(s) discussed herein, and no special significance is to be placed upon whether a term is elaborated or discussed herein. A recital of one or more synonyms does not exclude the use of other synonyms. The use of examples anywhere in this specification, including examples of any terms is illustrative only and in no way limits the scope and meaning of the present disclosure or of any exemplified term. Likewise, the present disclosure is not limited to various embodiments given herein. Numbering terms such as “first”, “second” or “third” can be used to describe various components, signals or the like, which are for distinguishing one component/signal from another one only, and are not intended to, nor should be construed to impose any substantive limitations on the components, signals or the like.

### First Embodiment

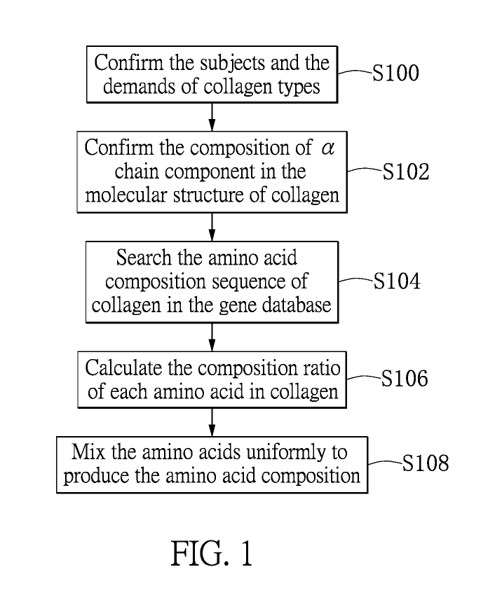

Refer to FIG. 1, which is a flowchart of preparing the amino acid composition for collagen formation according to an embodiment of the present disclosure. Firstly, the subjects and the demands of collagen types are confirmed (step S100). In the first embodiment, the subject is a human, and the demand is to supplement human type I collagen. It is worth mentioning that the amino acid composition for collagen formation of the present disclosure does not limit the subject to be administered; that is to say, the amino acid composition for collagen formation of the present disclosure can be supplied to other animals (for example cats and dogs, etc.) as needed, as long as the type of collagen is appropriately determined according to particular requirements. Therefore, the desired amino acid composition can be confirmed. For example, when the subject is a human, the collagen is selected from the group consisting of human type I collagen, human type II collagen, human type III collagen, human type IV collagen, human type V collagen, human type VI collagen, human type VII collagen, human type VID collagen, human type IX collagen, human type X collagen, human type X I collagen, human type X II collagen, human type X III collagen, human type X IV collagen, human type X V collagen, human type X VI collagen, human type X VII collagen, human type X VIII collagen, human type X IX collagen, human type X X collagen, human type X X I collagen, human type X X II collagen, human type X X III collagen, human type X X IV collagen, human type X X V collagen, human type X X VI collagen, human type X X VII collagen and human type X X VIII collagen. However, the present disclosure is not limited thereto.

Secondly, the composition of a chain component in the molecular structure of collagen is confirmed from relevant literature (refer to Advanced Drug Delivery Reviews 55 (2003) 1531-1546.) (step S102). It should be noted that, according to the results from consulting the relevant literature, the first chain unit component, the second chain unit component and the third chain unit component would be the same or different from each other; each component is selected from the group consisting of al chain component, α2 chain component, α3 chain component, α4 chain component, α5 chain component and α6 chain component. In detail, collagen is a helical elastic protein formed by three long-chain polypeptide chains (also called a chains) which are entangled in a left-handed manner. Each polypeptide chain is composed of a specific amino acid in a specific arrangement. For example, the amino acid composition for forming human type I collagen includes a first chain unit component, a second chain unit component and a third chain unit component, in which the first chain unit component and the second chain unit component are both al chain components, and the third chain unit component is an α2 chain component. On the other hand, the amino acid composition for forming human type II collagen includes a first chain unit component, a second chain unit component and a third chain unit component, in which the first chain unit component, second chain unit component, and third chain unit component are all al chain component.

Thirdly, the amino acid composition sequence of collagen in the gene database is searched (step S104). The amino acid composition sequence of collagen in the gene database does not contain modified amino acids after protein post-translational modification: namely, hydroxyproline and hydroxylysine. Therefore, it is possible to provide correct and appropriate various amino acids as raw materials for synthesizing collagen in human cells. In the first embodiment, the amino acid composition sequence of the al chain component and the α2 chain component of human type I collagen is queried using the NCBI gene database (https://www.ncbi.nlm.nih.gov/protein/). In the first embodiment, the type I collagen is composed of two al chain components and one α2 chain component; each of al chain component and α2 chain component includes 20 amino acids, which are as follows: Alanine, Phenylalanine, Cysteine, Aspartate, Asparagine, Glutamate, Glutamine, Glycine, Histidine, Leucine, Isoleucine, Lysine, Methionine, Proline, Arginine, Serine, Threonine, Valine, Tryptophan, and Tyrosine. According to this data, the total amount of each amino acid in the molecular structure of the type I collagen can be obtained. Thus it is possible to provide the correct and appropriate various amino acids as raw materials for synthesizing type I collagen in human cells. In addition, the kind of gene database to be queried is not limited in step S104.

Next, the composition ratio of each amino acid in collagen is calculated (step S106). Specifically, the weight percentage of each amino acid in the collagen is calculated based on 1 mole collagen. Based on the total weight of the amino acid composition, the amino acid composition for collagen formation includes: 0 to 5 weight percent (wt %) of Asparagine, 0 to 5 weight percent (wt %) of Cysteine, 0 to 5 weight percent (wt %) of Tyrosine, 0 to 10 weight percent (wt %) of Arginine, 0 to 10 weight percent (wt %) of Alanine, 0 to 10 weight percent (wt %) of Aspartate, 0 to 10 weight percent (wt %) of Glutamate, 0 to 10 weight percent (wt %) of Glutamine, 0 to 10 weight percent (wt %) of Serine, 0.01 to 10 weight percent (wt %) of Phenylalanine, 0.01 to 10 weight percent (wt %) of Valine, 0.01 to 10 weight percent (wt %) of Threonine, 0.01 to 10 weight percent (wt %) of Tryptophan, 0.01 to 10 weight percent (wt %) of Methionine, 0.01 to 10 weight percent (wt %) of Leucine, 0.01 to 10 weight percent (wt %) of Isoleucine, 0.01 to 10 weight percent (wt %) of Histidine, 0.01 to 10 weight percent (wt %) of Lysine, 10 to 25 weight percent (wt %) of Glycine and 10 to 25 weight percent (wt %) of Proline. In this example, the weight percentage of each amino acid in type I collagen was calculated based on 1 mole collagen.

Table 1 shows the components of the amino acid composition of the type I collagen of the first embodiment:

TABLE 1

| | | Molecular | Amount of | Amount of | | | |
|---|---|---|---|---|---|---|---|
| | Chemical | weight of | amino acids | amino acids | | | |
| Type of | formula of | amino acid | in α1 chain | in α2 chain | | | |
| amino acid | amino acid | (g/mol) | component | component | A | B | C |
| L-Isoleucine | C₆H₁₃NO₂ | 131.17 | 24 | 32 | 80 | 10,493.89 | 2.17% |
| L-Leucine | C₆H₁₃NO₂ | 131.17 | 48 | 61 | 157 | 20,594.26 | 4.25% |
| L-Lysine | C₆H₁₄N₂O₂ | 146.19 | 57 | 50 | 164 | 23,974.86 | 4.95% |
| L-Methionine | C₅H₁₁NO₂S | 149.21 | 13 | 10 | 36 | 5,371.65 | 1.11% |
| L-Phenylalanine | C₉H₁₁NO₂ | 165.19 | 27 | 22 | 76 | 12,554.44 | 2.59% |
| L-Threonine | C₄H₉NO₃ | 119.12 | 44 | 42 | 130 | 15,485.56 | 3.19% |
| L-Tryptophan | C₁₁H₁₂N₂O₂ | 204.23 | 6 | 5 | 17 | 3,471.85 | 0.72% |
| L-Valine | C₅H₁₁NO₂ | 117.15 | 47 | 55 | 149 | 17,454.89 | 3.60% |
| L-Histidine | C₆H₉N₃O₂ | 155.16 | 10 | 15 | 35 | 5,430.43 | 1.12% |
| L-Arginine | C₆H₁₄N₄O₂ | 174.20 | 72 | 72 | 216 | 37,627.57 | 7.76% |
| L-Alanine | C₃H₇NO₂ | 89.09 | 140 | 130 | 410 | 36,528.34 | 7.54% |
| L-Asparagine | C₄H₈N₂O₃ | 132.12 | 28 | 41 | 97 | 12,815.48 | 2.64% |
| L-Aspartate | C₄H₇NO₄ | 133.10 | 66 | 43 | 175 | 23,293.06 | 4.81% |
| L-Cysteine | C₃H₇NO₂S | 121.16 | 18 | 9 | 45 | 5,452.16 | 1.13% |
| L-Glutamate | C₅H₉NO₄ | 147.13 | 75 | 66 | 216 | 31,780.06 | 6.56% |
| L-Glutamine | C₅H₁₀N₂O₃ | 146.15 | 49 | 33 | 131 | 19,145.01 | 3.95% |
| Glycine | C₂H₅NO₂ | 75.07 | 391 | 381 | 1163 | 87,302.80 | 18.01% |
| L-Proline | C₅H₉NO₂ | 115.13 | 276 | 231 | 783 | 90,147.57 | 18.60% |
| L-Serine | C₃H₇NO₃ | 105.09 | 60 | 52 | 172 | 18,076.00 | 3.73% |
| L-Tyrosine | C₉H₁₁NO₃ | 181.19 | 13 | 16 | 42 | 7,609.95 | 1.57% |
| Sum | | | 1,464 | 1,366 | 4,294 | 484,609.82 | 100.00% |

A: amount of each amino acid in type I collagen = amount of such amino acid in α1 chain component × 2 + amount of such amino acid in α2 chain component (Human type I collagen includes a first chain unit component, a second chain unit component and a third chain unit component, wherein the first chain unit component and the second chain unit component are both α1 chain components, and the third chain unit component is α2 chain component.)
B: weight of each amino acid in type I collagen = amount of each amino acid in type I collagen (A) × amino acid molecular weight
C: weight percent of each amino acid in type I collagen = weight of the amino acid in type I collagen (B)/total weight of type I collagen × 100%

Based on the total weight of the amino acid composition for the first embodiment, the amino acid composition for forming human type I collagen includes the following: 2.17 wt % Isoleucine, 4.25 wt % Leucine, 4.95 wt % Lysine, 1.11 wt % Methionine, 2.59 wt % Phenylalanine, 3.19 wt % Threonine, 0.72 wt % Tryptophan, 3.60 wt % Valine, 1.12 wt % Histidine, 7.76 wt % Arginine, 7.54 wt % Alanine, 2.64 wt % Asparagine, 4.81 wt % Aspartate, 1.13 wt % Cysteine, 6.56 wt % Glutamate, 3.95 wt % Glutamine, 18.01 wt % Glycine, 18.60 wt % Proline, 3.73 wt % Serine and 1.57 wt % Tyrosine. However, the weight percent of each amino acid composition is not limited thereto.

Lastly, the amino acids are mixed uniformly to manufacture the amino acid composition (step S108). Specifically, each amino acid can be purchased commercially (for example, but not limited to being, purchased from Sigma-Aldrich). The pharmaceutically acceptable salt or ester derivatives of the above-mentioned amino acids can also be used alternatively, given that the above-mentioned esters of amino acids could be hydrolyzed by gastric acid to produce amino acids, and the above-mentioned salts of amino acids could produce amino acids in water. For example, L-Arginine ethyl ester is an ester of L-arginine, which would be hydrolyzed by gastric acid to produce L-arginine and ethanol. Further, L-Lysine Acetate is a salt of L-lysine and produces L-lysine and acetic acid in water. Similarly, L-Lysine Hydrochloride is a salt of L-lysine and produces L-lysine and hydrochloric acid in water.

### Second Embodiment

In the second embodiment, descriptions that are similar to those of the first embodiment will be omitted herein to avoid repetition. All of the contents in the first embodiment can be applied to the second embodiment. Only the main differences between the first embodiment and the second embodiment will be described below.

Reference is made to FIG. 1, which is a flowchart of preparing the amino acid composition. In step S100, the subject is a human, and the demand is to supplement human type II collagen. In step S102, the molecular structure of human type II collagen is confirmed with relevant literature (refer to Advanced Drug Delivery Reviews 55 (2003) 1531-1546.) to be composed of three al chain components. In step S104, a sequence of the al chain component of human type II collagen is queried in the NCBI gene database. In second embodiment, the human type II collagen is composed of al chain component. Further, each al chain component includes 20 amino acids, as with the first embodiment. Then, in step S106, the proportion of components of each of the amino acids in type II collagen is calculated. Based on the total weight of the amino acid composition, the amino acid composition for collagen formation includes the following: 0 to 5 weight percent (wt %) of Asparagine, 0 to 5 weight percent (wt %) of Cysteine, 0 to 5 weight percent (wt %) of Tyrosine, 0 to 10 weight percent (wt %) of Arginine, 0 to 10 weight percent (wt %) of Alanine, 0 to 10 weight percent (wt %) of Aspartate, 0 to 10 weight percent (wt %) of Glutamate, 0 to 10 weight percent (wt %) of Glutamine, 0 to 10 weight percent (wt %) of Serine, 0.01 to 10 weight percent (wt %) of Phenylalanine, 0.01 to 10 weight percent (wt %) of Valine, 0.01 to 10 weight percent (wt %) of Threonine, 0.01 to 10 weight percent (wt %) of Tryptophan, 0.01 to 10 weight percent (wt %) of Methionine, 0.01 to 10 weight percent (wt %) of Leucine, 0.01 to 10 weight percent (wt %) of Isoleucine, 0.01 to 10 weight percent (wt %) of Histidine, 0.01 to 10 weight percent (wt %) of Lysine, 10 to 25 weight percent (wt %) of Glycine and 10 to 25 weight percent (wt %) of Proline. In this example, the weight percentage of each amino acid in type II collagen is calculated based on 1 mole type II collagen.

Specifically, Table 2 shows the components of the amino acid composition of the type II collagen of the second embodiment:

TABLE 2

| | | Molecular | Amount of | | | |
|---|---|---|---|---|---|---|
| | Chemical | weight of | amino acids | | | |
| Type of | formula of | amino acid | in α1 chain | | | |
| amino acid | amino acid | (g/mol) | component | A | B | C |
| L-Isoleucine | C₆H₁₃NO₂ | 131.17 | 34 | 102 | 13,379.71 | 2.65% |
| L-Leucine | C₆H₁₃NO₂ | 131.17 | 56 | 168 | 22,037.16 | 4.36% |
| L-Lysine | C₆H₁₄N₂O₂ | 146.19 | 67 | 201 | 29,383.83 | 5.81% |
| L-Methionine | C₅H₁₁NO₂S | 149.21 | 16 | 48 | 7,162.20 | 1.41% |
| L-Phenylalanine | C₉H₁₁NO₂ | 165.19 | 25 | 75 | 12,389.25 | 2.45% |
| L-Threonine | C₄H₉NO₃ | 119.12 | 44 | 132 | 15,723.80 | 3.11% |
| L-Tryptophan | C₁₁H₁₂N₂O₂ | 204.23 | 7 | 21 | 4,288.75 | 0.85% |
| L-Valine | C₅H₁₁NO₂ | 117.15 | 38 | 114 | 13,354.75 | 2.64% |
| L-Histidine | C₆H₉N₃O₂ | 155.16 | 8 | 24 | 3,723.72 | 0.74% |
| L-Arginine | C₆H₁₄N₄O₂ | 174.20 | 72 | 216 | 37,627.57 | 7.44% |
| L-Alanine | C₃H₇NO₂ | 89.09 | 134 | 402 | 35,815.59 | 7.08% |
| L-Asparagine | C₄H₈N₂O₃ | 132.12 | 32 | 96 | 12,683.37 | 2.51% |
| L-Aspartate | C₄H₇NO₄ | 133.10 | 62 | 186 | 24,757.20 | 4.90% |
| L-Cysteine | C₃H₇NO₂S | 121.16 | 19 | 57 | 6,906.06 | 1.37% |
| L-Glutamate | C₅H₉NO₄ | 147.13 | 79 | 237 | 34,869.79 | 6.90% |
| L-Glutamine | C₅H₁₀N₂O₃ | 146.15 | 60 | 180 | 26,306.12 | 5.20% |
| Glycine | C₂H₅NO₂ | 75.07 | 406 | 1,218 | 91,431.48 | 18.08% |
| L-Proline | C₅H₉NO₂ | 115.13 | 270 | 810 | 93,256.11 | 18.44% |
| L-Serine | C₃H₇NO₃ | 105.09 | 48 | 144 | 15,133.39 | 2.99% |
| L-Tyrosine | C₉H₁₁NO₃ | 181.19 | 10 | 30 | 5,435.68 | 1.07% |
| Sum | | | 1,487 | 4,461 | 505,665.52 | 100.00% |

A: amount of each amino acid in type II collagen = amount of such amino acid in α1 chain component × 3 (Human type II collagen includes a first chain unit component, a second chain unit component and a third chain unit component, wherein the first chain unit component and the second chain unit and a third chain unit component are all α1 chain components.)
B: weight of each amino acid in type II collagen = amount of each amino acid in type II collagen (A) × amino acid molecular weight
C: weight percent of each amino acid in type II collagen = weight of the amino acid in type II collagen (B)/total weight of type II collagen × 100%

Therefore, in the second embodiment, based on the total weight of the amino acid composition, the amino acid composition for forming human type II collagen includes the following: 2.65 wt % Isoleucine, 4.36 wt % Leucine, 5.81 wt % Lysine, 1.41 wt % Methionine, 2.45 wt % Phenylalanine, 3.11 wt % Threonine, 0.85 wt % Tryptophan, 2.64 wt % Valine, 0.74 wt % Histidine, 7.44 wt % Arginine, 7.08 wt % Alanine, 2.51 wt % Asparagine, 4.90 wt % Aspartate, 1.37 wt % Cysteine, 6.90 wt % Glutamate, 5.20 wt % Glutamine, 18.08 wt % Glycine, 18.44 wt % Proline, 2.99 wt % Serine and 1.07 wt % Tyrosine. However, the weight percentage of each amino acid composition is not limited thereto.

Lastly, the amino acids are mixed uniformly to manufacture the amino acid composition (step S108). The amino acid composition of the first embodiment and the second embodiment may further optionally include a cofactor component, which is a non-protein compound binding to an enzyme, e.g. vitamin or metal ion. Furthermore, the vitamin is selected from the following group: vitamin C, vitamin B1 and vitamin A; the metal ion is selected from the group consisting of copper ion, magnesium ion and zinc ion. However, the present disclosure is not limited thereto.

In addition, the amino acid composition of the present disclosure is suitable in providing food products (functional food, special purpose foods, specified health food, dietary supplements, foods for diet therapy, health foods, nutritional supplements, etc.). The food products may be a health food in the form of a tablet, a capsule, a powder, a granule, a beverage (including a solution and a suspension), or a refreshing beverage, a seasoning, or a processed food. However, the present disclosure is not limited thereto.

In conclusion, an advantage of the present disclosure is that the amino acid composition for collagen formation can utilize the technical features of “a first chain unit component, a second chain unit component and a third chain unit component, each being selected from the group consisting of al chain component, α2 chain component, α3 chain component, α4 chain component, α5 chain component and α6 chain component” and “al chain component, α2 chain component, α3 chain component, α4 chain component, α5 chain component and α6 chain component, each including Alanine, Phenylalanine, Cysteine, Aspartate, Asparagine, Glutamate, Glutamine, Glycine, Histidine, Leucine, Isoleucine, Lysine, Methionine, Proline, Arginine, Serine, Threonine, Valine, Tryptophan, and Tyrosine and/or the pharmaceutically acceptable salt or ester derivatives thereof” to achieve the beneficial effect of the present disclosure.

Specifically, the amino acid composition of the present disclosure can be directly sent to the bloodstream through the small intestine, and then sent to other tissue cells without gastrointestinal digestion. The amino acid composition can be absorbed directly to form collagen. Furthermore, the amino acid composition of the present disclosure is to synthesize collagen directly by amino acids and not by collagen or collagen peptide extracted from animal sources. Therefore, the amino acid composition of the present disclosure can avoid allergies in consumers who are allergic to specific sources and is suitable for ingestion by vegetarians.

Moreover, the amino acid composition for collagen formation of the present disclosure can target a specific subject and the requisite type of collagen to determine appropriate amino acid composition, as well as the specific ratio. The amino acid composition can be absorbed directly and utilized by organisms to achieve the purpose of synthesizing collagen.

The foregoing description of the exemplary embodiments of the disclosure has been presented only for the purposes of illustration and description and is not intended to be exhaustive or to limit the disclosure to the precise forms disclosed. Many modifications and variations are possible in light of the above teaching.

The embodiments were chosen and described in order to explain the principles of the disclosure and their practical application so as to enable others skilled in the art to utilize the disclosure and various embodiments and with various modifications as are suited to the particular use contemplated. Alternative embodiments will become apparent to those skilled in the art to which the present disclosure pertains without departing from its spirit and scope.

What is claimed is:

1. An amino acid composition for collagen formation, comprising:
a first chain unit component;
a second chain unit component; and
a third chain unit component;
wherein the first chain unit component, the second chain unit component and the third chain unit component are the same or different from each other, and each is selected from the group consisting of al chain component, α2 chain component, α3 chain component, α4 chain component, α5 chain component and α6 chain component;
wherein the al chain component, α2 chain component, α3 chain component, α4 chain component, α5 chain component and α6 chain component, and each includes Alanine, Phenylalanine, Cysteine, Aspartate, Asparagine, Glutamate, Glutamine, Glycine, Histidine, Leucine, Isoleucine, Lysine, Methionine, Proline, Arginine, Serine, Threonine, Valine, Tryptophan, and Tyrosine and/or the pharmaceutically acceptable salt or ester derivatives thereof.

2. The amino acid composition for collagen formation according to claim 1,
wherein the collagen is selected from the group consisting of human type I collagen, human type II collagen, human type III collagen, human type IV collagen, human type V collagen, human type VI collagen, human type VII collagen, human type VIII collagen, human type IX collagen, human type X collagen, human type X I collagen, human type X II collagen, human type X III collagen, human type X IV collagen, human type X V collagen, human type X VI collagen, human type X VII collagen, human type X VIII collagen, human type X IX collagen, human type X X collagen, human type X X I collagen, human type X X II collagen, human type X X III collagen, human type X X IV collagen, human type X X V collagen, human type X X VI collagen, human type X X VII collagen and human type X X VIII collagen.

3. The amino acid composition for collagen formation according to claim 2, wherein based on the total weight of the amino acid composition, the amino acid composition includes:
0 to 5 weight percent (wt %) of Asparagine;
0 to 5 weight percent (wt %) of Cysteine;
0 to 5 weight percent (wt %) of Tyrosine;
0 to 10 weight percent (wt %) of Arginine;
0 to 10 weight percent (wt %) of Alanine;
0 to 10 weight percent (wt %) of Aspartate;
0 to 10 weight percent (wt %) of Glutamate;
0 to 10 weight percent (wt %) of Glutamine;
0 to 10 weight percent (wt %) of Serine;
0.01 to 10 weight percent (wt %) of Phenylalanine;
0.01 to 10 weight percent (wt %) of Valine;
0.01 to 10 weight percent (wt %) of Threonine;
0.01 to 10 weight percent (wt %) of Tryptophan;
0.01 to 10 weight percent (wt %) of Methionine;
0.01 to 10 weight percent (wt %) of Leucine;
0.01 to 10 weight percent (wt %) of Isoleucine;
0.01 to 10 weight percent (wt %) of Histidine;
0.01 to 10 weight percent (wt %) of Lysine;
10 to 25 weight percent (wt %) of Glycine; and
10 to 25 weight percent (wt %) of Proline.

4. The amino acid composition for collagen formation according to claim 2, wherein when the collagen is human type I collagen, the first chain unit component and the second chain unit component are both al chain components, and the third chain unit component is an α2 chain component; wherein the amino acid composition includes: 2.17 wt % Isoleucine, 4.25 wt % Leucine, 4.95 wt % Lysine, 1.11 wt % Methionine, 2.59 wt % Phenylalanine, 3.19 wt % Threonine, 0.72 wt % Tryptophan, 3.60 wt % Valine, 1.12 wt % Histidine, 7.76 wt % Arginine, 7.54 wt % Alanine, 2.64 wt % Asparagine, 4.81 wt % Aspartate, 1.13 wt % Cysteine, 6.56 wt % Glutamate, 3.95 wt % Glutamine, 18.01 wt % Glycine, 18.60 wt % Proline, 3.73 wt % Serine and 1.57 wt % Tyrosine.

5. The amino acid composition for collagen formation according to claim 2, wherein when the collagen is human type II collagen, the first chain unit component, the second chain unit component, and the third chain unit component are all al chain components; wherein the amino acid composition includes: 2.65 wt % Isoleucine, 4.36 wt % Leucine, 5.81 wt % Lysine, 1.41 wt % Methionine, 2.45 wt % Phenylalanine, 3.11 wt % Threonine, 0.85 wt % Tryptophan, 2.64 wt % Valine, 0.74 wt % Histidine, 7.44 wt % Arginine, 7.08 wt % Alanine, 2.51 wt % Asparagine, 4.90 wt % Aspartate, 1.37 wt % Cysteine, 6.90 wt % Glutamate, 5.20 wt % Glutamine, 18.08 wt % Glycine, 18.44 wt % Proline, 2.99 wt % Serine and 1.07 wt % Tyrosine.

6. The amino acid composition for collagen formation according to claim 2, further comprising: a cofactor component, wherein the cofactor component is selected from the group consisting of vitamin C, vitamin B1, vitamin A, copper ion, magnesium ion, and zinc ion.

7. A method for supplementing collagen to a subject, comprising: providing the amino acid composition for collagen formation according to claim 1 to a subject; wherein the subject is a vegetarian or a human having the symptoms of an allergy originating from animal protein.
